(19) United States
(12) Patent Application Publication
(10) Pub. No.: US 2018/0015128 A1
(43) Pub. Date: Jan. 18, 2018

(54) Amniotic Fluid-Derived Preparation with a Standardized Biologic Activity

(71) Applicant: Applied Biologics, LLC, Scottsdale, AZ (US)

(72) Inventor: Edward Britt, Scottsdale, AZ (US)

(21) Appl. No.: 15/637,958

(22) Filed: Jun. 29, 2017

Related U.S. Application Data
(60) Provisional application No. 62/356,315, filed on Jun. 29, 2016.

(51) Int. Cl. A61K 35/50; A61L 27/36; A61K 38/18; C07K 14/475; C12N 5/00

(52) U.S. Cl. CPC A61K 35/50; C07K 14/475; C12N 5/0018; A61K 38/18; A61L 27/3604; C12N 2501/10

(57) ABSTRACT

This invention relates to preparations derived from amniotic fluid, and more specifically, to an amniotic fluid preparation with specific standardized measure of the biologic activity of a component of the preparation, such as a constituent protein, for use in clinical and research applications. The amniotic fluid preparation with standardized biologic activity resolves problems of establishing efficacy, safety, and reproducibility in the clinical and investigatory use of amniotic fluid-derived preparations for the treatment of a wide range of degenerative illnesses, traumatic injuries, tissue and organ transplantation, and other medical conditions.

### CROSS-REFERENCE TO RELATED APPLICATIONS

This application claims the benefit of and priority to U.S. Provisional Patent Application No. 62/356,315, filed Jun. 29, 2016, the entire contents of which are incorporated herein by reference.

### BACKGROUND OF THE INVENTION

### Technical Field

This invention relates to preparations derived from amniotic fluid, and, more specifically, to amniotic fluid-derived preparations with a standardized biologic activity particular to biologically active components, such as proteins, for use in clinical medicine and research applications.

### State of the Art

Amniotic fluid, specifically human amniotic fluid, has been identified as a rich source of therapeutic biomolecules. Amniotic fluid's suspended protein fraction contains a complex biological soup of growth factors, inflammatory regulators, immuno-modulators, and other active biomolecules. Amniotic fluid is also rich in pluripotent cellular elements, including amniotic fluid stem cells which in turn contain high intracellular concentrations of regulatory proteins and other biologically active substances. Following cellular lysis, these intracellular proteins and other bioactive substances are available within an amniotic fluid-derived preparation. Absent cellular death and lysis, intact, viable cells may secrete proteins and bioactive substances into the extracellular milieu, creating additional therapeutically relevant bioactivity.

Amniotic fluid-derived compositions that are processed and concentrated in a manner which preserves bioactivity, quantified with respect to specific proteins and other bioactive compounds, and then packaged for convenient and practical use by a clinician or research scientist are not currently available. Amniotic fluid-derived compositions that are not standardized for clinical use—including clinical trials in human subjects—may lead to unpredictable results or be unsafe. Additionally, an unstandardized composition is problematic for researchers to obtain reproducible results in research applications.

Accordingly, what is needed are amniotic fluid-derived compositions with a standardized biologic activity for clinical and investigational use.

Citation of documents herein is not an admission by the applicant that any is pertinent prior art. Stated dates or representation of the contents of any document is based on the information available to the applicant and does not constitute any admission of the correctness of the dates or contents of any document.

### SUMMARY OF THE INVENTION

Disclosed is an amniotic fluid derivative comprising a supernatant separated from a donor amniotic fluid; and a fluid, wherein the fluid dilutes the supernatant to a standardized biologic activity in the amniotic fluid preparation.

In some embodiments, the amniotic fluid derivative further comprises a cellular lysate. In some embodiments, the cellular lysate is an amniotic fluid cellular component cellular lysate. In some embodiments, the amniotic fluid derivative further comprises a platelet-rich plasma. In some embodiments, the amniotic fluid derivative further comprises a recombinant protein. In some embodiments, the amniotic fluid derivative further comprises a culture media sourced from a stem cell culture, wherein the culture media comprises a secondary source protein.

Disclosed is an amniotic fluid derivative with a standardized biologic activity comprising a first protein fraction comprising one or more proteins separated from a donor amniotic fluid; and a fluid, wherein the fluid dilutes the first protein fraction to a standardized concentration in the amniotic fluid preparation.

In some embodiments, the first protein fraction substantially comprises an epidermal growth factor. In some embodiments, the concentration of the epidermal growth factor is greater than about 20 picograms per milliliter (“pg/ml”). In some embodiments, the first protein fraction substantially comprises angiogenin. In some embodiments, the concentration of angiogenin is greater than about 300 picograms per milliliter. In some embodiments, the first protein fraction substantially comprises monocyte chemoattractant protein-1. In some embodiments, the concentration of monocyte chemoattractant protein-1 is greater than about 10 picograms per milliliter. In some embodiments, the first protein fraction substantially comprises matrix metalloproteinase-1. In some embodiments, the first protein fraction substantially comprises matrix metalloproteinase-10. in some embodiments, the concentration of matrix metalloproteinase-10 is greater than about 300 picograms per milliliter. In some embodiments, and first protein fraction substantially comprises tissue inhibitor of matrix metalloproteinase-1. In some embodiments, the concentration of tissue inhibitor of matrix metalloproteinase-1 is greater than about 4,000 picograms per milliliter. In some embodiments, the first protein fraction substantially comprises tissue inhibitor of matrix metalloproteinase-2. In some embodiments, the concentration of the tissue inhibitor of matrix metalloproteinase-2 is greater than about 1,500 picograms per milliliter. In some embodiments, the first protein fraction substantially comprises hepatocyte growth factor. In some embodiments, the concentration of hepatocyte growth factor is greater than about 200 picograms per milliliter. In some embodiments, the first protein fraction substantially comprises insulin-like growth factor binding protein-2. In some embodiments, the concentration of insulin-like growth factor binding protein-2 is greater than about 6,000 picograms per milliliter.

In some embodiments, a first protein fraction substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is about 1 pg/ml, about 5 pg/ml, about 10 pg/ml, about 15 pg/ml, about 20 pg/ml, about 25 pg/ml, about 30 pg/ml, about 40 pg/ml, about 50 pg/ml, about 75 pg/ml, about 100 pg/ml, about 125 pg/ml, about 150 pg/ml, about 175 pg/ml, about 200 pg/ml, about 225 pg/ml, about 250 pg/ml, about 275 pg/ml, about 300 pg/ml, about 325 pg/ml, about 350 pg/ml, about 375 pg/ml, about 400 pg/ml, about 500 pg/ml, about 600 pg/ml, about 700 pg/ml, about 800 pg/ml, about 900 pg/ml, about 1,000 pg/ml, about 1,100 pg/ml, about 1,200 pg/ml, about 1,300 pg/ml, about 1,400 pg/ml, about 1,500 pg/ml, about 1,600 pg/ml, about 1,700 pg/ml, about 1,800 pg/ml, about 1,900 pg/ml, about 2,000 pg/ml, about 2,500 pg/ml, about 3,000 pg/ml, about 3,500 pg/ml, about 4,000 pg/ml, about 4,500 pg/ml, about 5,000 pg/ml, about 5,500 pg/ml, about 6,000 pg/ml, about 6,500 pg/ml, about 7,000 pg/ml, about 7,500 pg/ml, about 8,000 pg/ml, about 8,500 pg/ml, about 9,000 pg/ml, about 9,500 pg/ml, or about 10,000 pg/ml.

In some embodiments, a first protein fraction substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is greater than 1 pg/ml, greater than 5 pg/ml, greater than 10 pg/ml, greater than 15 pg/ml, greater than 20 pg/ml, greater than 25 pg/ml, greater than 30 pg/ml, greater than 40 pg/ml, greater than 50 pg/ml, greater than 75 pg/ml, greater than 100 pg/ml, greater than 125 pg/ml, greater than 150 pg/ml, greater than 175 pg/ml, greater than 200 pg/ml, greater than 225 pg/ml, greater than 250 pg/ml, greater than 275 pg/ml, greater than 300 pg/ml, greater than 325 pg/ml, greater than 350 pg/ml, greater than 375 pg/ml, greater than 400 pg/ml, greater than 500 pg/ml, greater than 600 pg/ml, greater than 700 pg/ml, greater than 800 pg/ml, greater than 900 pg/ml, greater than 1,000 pg/ml, greater than 1,100 pg/ml, greater than 1,200 pg/ml, greater than 1,300 pg/ml, greater than 1,400 pg/ml, greater than 1,500 pg/ml, greater than 1,600 pg/ml, greater than 1,700 pg/ml, greater than 1,800 pg/ml, greater than 1,900 pg/ml, greater than 2,000 pg/ml, greater than 2,500 pg/ml, greater than 3,000 pg/ml, greater than 3,500 pg/ml, greater than 4,000 pg/ml, greater than 4,500 pg/ml, greater than 5,000 pg/ml, greater than 5,500 pg/ml, greater than 6,000 pg/ml, greater than 6,500 pg/ml, greater than 7,000 pg/ml, greater than 7,500 pg/ml, greater than 8,000 pg/ml, greater than 8,500 pg/ml, greater than 9,000 pg/ml, greater than 9,500 pg/ml, or greater than 10,000 pg/ml.

In some embodiments, a first protein fraction substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is between about 1 pg/ml and about 10 pg/ml, between about 10 pg/ml and about 20 pg/ml, between about 20 pg/ml and about 40 pg/ml, between about 40 pg/ml and about 60 pg/ml, between about 60 pg/ml and about 80 pg/ml, between about 80 pg/ml and about 100 pg/ml, between about 100 pg/ml and about 150 pg/ml, between about 150 pg/ml and about 200 pg/ml, between about 200 pg/ml and about 250 pg/ml, between about 250 pg/ml and about 300 pg/ml, between about 300 pg/ml and about 350 pg/ml, between about 350 pg/ml and about 400 pg/ml, between about 400 pg/ml and about 500 pg/ml, between about 500 pg/ml and about 600 pg/ml, between about 600 pg/ml and about 700 pg/ml, between about 700 pg/ml and about 800 pg/ml, between about 800 pg/ml and about 900 pg/ml, between about 900 pg/ml and about 1,000 pg/ml, between about 1,000 pg/ml and about 1,500 pg/ml, between about 1,500 pg/ml and about 2,000 pg/ml, between about 2,500 pg/ml and about 3,000 pg/ml, between about 3,000 pg/ml and about 3,500 pg/ml, between about 3,500 pg/ml and about 4,000 pg/ml, between about 4,000 pg/ml and about 4,500 pg/ml, between about 4,500 pg/ml and about 5,000 pg/ml, between about 5,000 pg/ml and about 5,500 pg/ml, between about 5,500 pg/ml and about 6,000 pg/ml, between about 6,000 pg/ml and about 6,500 pg/ml, between about 6,500 pg/ml and about 7,000 pg/ml, between about 7,000 pg/ml and about 7,500 pg/ml, between about 7,500 pg/ml and about 8,000 pg/ml, between about 8,000 pg/ml and about 8,500 pg/ml, between about 8,500 pg/ml and about 9,000 pg/ml, between about 9,000 pg/ml and about 9,500 pg/ml, or between about 9,500 pg/ml and about 10,000 pg/ml.

Disclosed is an amniotic fluid derivative comprising a supernatant separated from a donor amniotic fluid; a second protein fraction separated from a secondary protein source; and a fluid, wherein the fluid dilutes the secondary protein fraction to a designated or standardized concentration in the amniotic fluid preparation.

In some embodiments, the secondary source is a cellular lysate. In some embodiments, the cellular lysate comprises an amniotic fluid cellular component. In some embodiments, the second protein fraction comprises a recombinant protein. In some embodiments, the secondary source is a platelet-rich plasma. In some embodiments, amniotic fluid derivative comprises a culture media sourced from a stem-cell culture, wherein the culture media comprises the secondary source.

In some embodiments, the invention includes a set of amniotic fluid preparations or a set of amniotic fluid derivatives. In some embodiments, the invention includes a set of amniotic fluid preparations, wherein each amniotic fluid preparation in the set comprises a supernatant separated from a donor amniotic fluid and a fluid, wherein the fluid dilutes the supernatant to a standardized biologic activity in the amniotic fluid preparation. In such embodiments, each amniotic fluid preparation included in a set of amniotic fluid preparations has the same or about the same standardized biologic activity as every other amniotic fluid preparation in the set.

In some embodiments, the invention includes a set of amniotic fluid preparations, wherein each amniotic fluid preparation in the set comprises a first protein fraction comprising one or more proteins separated from a donor amniotic fluid, and a fluid, wherein the fluid dilutes the first protein fraction to a standardized concentration in the amniotic fluid preparation. In such embodiments, the standardized concentration of the first protein fraction in each amniotic fluid preparation included in the set of amniotic fluid preparations is the same or about the same.

In some embodiments, a first protein fraction in each amniotic fluid preparation comprising a set of amniotic fluid preparations substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is about 1 pg/ml, about 5 pg/ml, about 10 pg/ml, about 15 pg/ml, about 20 pg/ml, about 25 pg/ml, about 30 pg/ml, about 40 pg/ml, about 50 pg/ml, about 75 pg/ml, about 100 pg/ml, about 125 pg/ml, about 150 pg/ml, about 175 pg/ml, about 200 pg/ml, about 225 pg/ml, about 250 pg/ml, about 275 pg/ml, about 300 pg/ml, about 325 pg/ml, about 350 pg/ml, about 375 pg/ml, about 400 pg/ml, about 500 pg/ml, about 600 pg/ml, about 700 pg/ml, about 800 pg/ml, about 900 pg/ml, about 1,000 pg/ml, about 1,100 pg/ml, about 1,200 pg/ml, about 1,300 pg/ml, about 1,400 pg/ml, about 1,500 pg/ml, about 1,600 pg/ml, about 1,700 pg/ml, about 1,800 pg/ml, about 1,900 pg/ml, about 2,000 pg/ml, about 2,500 pg/ml, about 3,000 pg/ml, about 3,500 pg/ml, about 4,000 pg/ml, about 4,500 pg/ml, about 5,000 pg/ml, about 5,500 pg/ml, about 6,000 pg/ml, about 6,500 pg/ml, about 7,000 pg/ml, about 7,500 pg/ml, about 8,000 pg/ml, about 8,500 pg/ml, about 9,000 pg/ml, about 9,500 pg/ml, or about 10,000 pg/ml.

In some embodiments, a first protein fraction in each amniotic fluid preparation comprising a set of amniotic fluid preparations substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is greater than 1 pg/ml, greater than 5 pg/ml, greater than 10 pg/ml, greater than 15 pg/ml, greater than 20 pg/ml, greater than 25 pg/ml, greater than 30 pg/ml, greater than 40 pg/ml, greater than 50 pg/ml, greater than 75 pg/ml, greater than 100 pg/ml, greater than 125 pg/ml, greater than 150 pg/ml, greater than 175 pg/ml, greater than 200 pg/ml, greater than 225 pg/ml, greater than 250 pg/ml, greater than 275 pg/ml, greater than 300 pg/ml, greater than 325 pg/ml, greater than 350 pg/ml, greater than 375 pg/ml, greater than 400 pg/ml, greater than 500 pg/ml, greater than 600 pg/ml, greater than 700 pg/ml, greater than 800 pg/ml, greater than 900 pg/ml, greater than 1,000 pg/ml, greater than 1,100 pg/ml, greater than 1,200 pg/ml, greater than 1,300 pg/ml, greater than 1,400 pg/ml, greater than 1,500 pg/ml, greater than 1,600 pg/ml, greater than 1,700 pg/ml, greater than 1,800 pg/ml, greater than 1,900 pg/ml, greater than 2,000 pg/ml, greater than 2,500 pg/ml, greater than 3,000 pg/ml, greater than 3,500 pg/ml, greater than 4,000 pg/ml, greater than 4,500 pg/ml, greater than 5,000 pg/ml, greater than 5,500 pg/ml, greater than 6,000 pg/ml, greater than 6,500 pg/ml, greater than 7,000 pg/ml, greater than 7,500 pg/ml, greater than 8,000 pg/ml, greater than 8,500 pg/ml, greater than 9,000 pg/ml, greater than 9,500 pg/ml, or greater than 10,000 pg/ml.

In some embodiments, a first protein fraction in each amniotic fluid preparation comprising a set of amniotic fluid preparations substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is between about 1 pg/ml and about 10 pg/ml, between about 10 pg/ml and about 20 pg/ml, between about 20 pg/ml and about 40 pg/ml, between about 40 pg/ml and about 60 pg/ml, between about 60 pg/ml and about 80 pg/ml, between about 80 pg/ml and about 100 pg/ml, between about 100 pg/ml and about 150 pg/ml, between about 150 pg/ml and about 200 pg/ml, between about 200 pg/ml and about 250 pg/ml, between about 250 pg/ml and about 300 pg/ml, between about 300 pg/ml and about 350 pg/ml, between about 350 pg/ml and about 400 pg/ml, between about 400 pg/ml and about 500 pg/ml, between about 500 pg/ml and about 600 pg/ml, between about 600 pg/ml and about 700 pg/ml, between about 700 pg/ml and about 800 pg/ml, between about 800 pg/ml and about 900 pg/ml, between about 900 pg/ml and about 1,000 pg/ml, between about 1,000 pg/ml and about 1,500 pg/ml, between about 1,500 pg/ml and about 2,000 pg/ml, between about 2,500 pg/ml and about 3,000 pg/ml, between about 3,000 pg/ml and about 3,500 pg/ml, between about 3,500 pg/ml and about 4,000 pg/ml, between about 4,000 pg/ml and about 4,500 pg/ml, between about 4,500 pg/ml and about 5,000 pg/ml, between about 5,000 pg/ml and about 5,500 pg/ml, between about 5,500 pg/ml and about 6,000 pg/ml, between about 6,000 pg/ml and about 6,500 pg/ml, between about 6,500 pg/ml and about 7,000 pg/ml, between about 7,000 pg/ml and about 7,500 pg/ml, between about 7,500 pg/ml and about 8,000 pg/ml, between about 8,000 pg/ml and about 8,500 pg/ml, between about 8,500 pg/ml and about 9,000 pg/ml, between about 9,000 pg/ml and about 9,500 pg/ml, or between about 9,500 pg/ml and about 10,000 pg/ml.

In some embodiments, the invention includes a set of amniotic fluid derivatives, wherein each amniotic fluid derivative comprises a supernatant separated from a donor amniotic fluid, a protein fraction separated from a secondary protein source, and a fluid, wherein the fluid dilutes the protein fraction from the secondary protein source to a designated or standardized concentration in the amniotic fluid preparation. In such embodiments, the designated concentration of the protein from the secondary protein source of each amniotic fluid derivative included in the set of amniotic fluid derivatives is the same or about the same.

In various embodiments, the invention may comprise a set of amniotic fluid preparations or amniotic fluid derivatives that includes a minimum number of amniotic fluid preparations or amniotic fluid derivatives. For instance, a set of amniotic fluid preparations or amniotic fluid derivatives of the invention may include at least 1, at least 5, at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, or at least 1000 amniotic fluid preparations or amniotic fluid derivatives.

Also described herein are methods of forming amniotic fluid derivatives. In some embodiments, a method of forming an amniotic fluid preparation includes the steps of separating an amniotic fluid supernatant from a donor amniotic fluid and diluting the supernatant to a standardized biologic activity with a fluid. In some embodiments, the amniotic fluid supernatant is separated from the donor amniotic fluid by centrifugation. In some embodiments, the amniotic fluid supernatant is separated from the donor amniotic fluid by any of centrifugation, precipitation, filtration, or a combination thereof. In some embodiments, the method of forming an amniotic fluid preparation includes the step of adding one or more of a cellular lysate, a recombinant protein, a platelet-rich plasma, or a stem cell culture medium.

The invention also includes methods of forming an amniotic fluid preparation that includes the steps of separating a first amniotic fluid protein fraction from a donor amniotic fluid and diluting the first amniotic fluid protein fraction to a standardized concentration with a liquid. In some embodiments, the first amniotic fluid protein fraction is separated from the donor amniotic fluid by centrifugation. In some embodiments, the first amniotic fluid protein fraction is separated from the donor amniotic fluid by any of centrifugation, precipitation, filtration, or a combination thereof.

In some embodiments of the foregoing method, the first amniotic fluid protein fraction substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is about 1 pg/ml, about 5 pg/ml, about 10 pg/ml, about 15 pg/ml, about 20 pg/ml, about 25 pg/ml, about 30 pg/ml, about 40 pg/ml, about 50 pg/ml, about 75 pg/ml, about 100 pg/ml, about 125 pg/ml, about 150 pg/ml, about 175 pg/ml, about 200 pg/ml, about 225 pg/ml, about 250 pg/ml, about 275 pg/ml, about 300 pg/ml, about 325 pg/ml, about 350 pg/ml, about 375 pg/ml, about 400 pg/ml, about 500 pg/ml, about 600 pg/ml, about 700 pg/ml, about 800 pg/ml, about 900 pg/ml, about 1,000 pg/ml, about 1,100 pg/ml, about 1,200 pg/ml, about 1,300 pg/ml, about 1,400 pg/ml, about 1,500 pg/ml, about 1,600 pg/ml, about 1,700 pg/ml, about 1,800 pg/ml, about 1,900 pg/ml, about 2,000 pg/ml, about 2,500 pg/ml, about 3,000 pg/ml, about 3,500 pg/ml, about 4,000 pg/ml, about 4,500 pg/ml, about 5,000 pg/ml, about 5,500 pg/ml, about 6,000 pg/ml, about 6,500 pg/ml, about 7,000 pg/ml, about 7,500 pg/ml, about 8,000 pg/ml, about 8,500 pg/ml, about 9,000 pg/ml, about 9,500 pg/ml, or about 10,000 pg/ml.

In some embodiments of the foregoing method, the first amniotic fluid protein fraction substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is greater than 1 pg/ml, greater than 5 pg/ml, greater than 10 pg/ml, greater than 15 pg/ml, greater than 20 pg/ml, greater than 25 pg/ml, greater than 30 pg/ml, greater than 40 pg/ml, greater than 50 pg/ml, greater than 75 pg/ml, greater than 100 pg/ml, greater than 125 pg/ml, greater than 150 pg/ml, greater than 175 pg/ml, greater than 200 pg/ml, greater than 225 pg/ml, greater than 250 pg/ml, greater than 275 pg/ml, greater than 300 pg/ml, greater than 325 pg/ml, greater than 350 pg/ml, greater than 375 pg/ml, greater than 400 pg/ml, greater than 500 pg/ml, greater than 600 pg/ml, greater than 700 pg/ml, greater than 800 pg/ml, greater than 900 pg/ml, greater than 1,000 pg/ml, greater than 1,100 pg/ml, greater than 1,200 pg/ml, greater than 1,300 pg/ml, greater than 1,400 pg/ml, greater than 1,500 pg/ml, greater than 1,600 pg/ml, greater than 1,700 pg/ml, greater than 1,800 pg/ml, greater than 1,900 pg/ml, greater than 2,000 pg/ml, greater than 2,500 pg/ml, greater than 3,000 pg/ml, greater than 3,500 pg/ml, greater than 4,000 pg/ml, greater than 4,500 pg/ml, greater than 5,000 pg/ml, greater than 5,500 pg/ml, greater than 6,000 pg/ml, greater than 6,500 pg/ml, greater than 7,000 pg/ml, greater than 7,500 pg/ml, greater than 8,000 pg/ml, greater than 8,500 pg/ml, greater than 9,000 pg/ml, greater than 9,500 pg/ml, or greater than 10,000 pg/ml.

In some embodiments of the foregoing method, the first amniotic fluid protein fraction substantially comprises any of an epidermal growth factor, angiogenin, monocyte chemoattractant protein-1, matrix metalloproteinase-1, matrix metalloproteinase-10, tissue inhibitor of matrix metalloproteinase-1, tissue inhibitor of matrix metalloproteinase-2, hepatocyte growth factor, or insulin-like growth factor binding protein-2, and the concentration of said protein is between about 1 pg/ml and about 10 pg/ml, between about 10 pg/ml and about 20 pg/ml, between about 20 pg/ml and about 40 pg/ml, between about 40 pg/ml and about 60 pg/ml, between about 60 pg/ml and about 80 pg/ml, between about 80 pg/ml and about 100 pg/ml, between about 100 pg/ml and about 150 pg/ml, between about 150 pg/ml and about 200 pg/ml, between about 200 pg/ml and about 250 pg/ml, between about 250 pg/ml and about 300 pg/ml, between about 300 pg/ml and about 350 pg/ml, between about 350 pg/ml and about 400 pg/ml, between about 400 pg/ml and about 500 pg/ml, between about 500 pg/ml and about 600 pg/ml, between about 600 pg/ml and about 700 pg/ml, between about 700 pg/ml and about 800 pg/ml, between about 800 pg/ml and about 900 pg/ml, between about 900 pg/ml and about 1,000 pg/ml, between about 1,000 pg/ml and about 1,500 pg/ml, between about 1,500 pg/ml and about 2,000 pg/ml, between about 2,500 pg/ml and about 3,000 pg/ml, between about 3,000 pg/ml and about 3,500 pg/ml, between about 3,500 pg/ml and about 4,000 pg/ml, between about 4,000 pg/ml and about 4,500 pg/ml, between about 4,500 pg/ml and about 5,000 pg/ml, between about 5,000 pg/ml and about 5,500 pg/ml, between about 5,500 pg/ml and about 6,000 pg/ml, between about 6,000 pg/ml and about 6,500 pg/ml, between about 6,500 pg/ml and about 7,000 pg/ml, between about 7,000 pg/ml and about 7,500 pg/ml, between about 7,500 pg/ml and about 8,000 pg/ml, between about 8,000 pg/ml and about 8,500 pg/ml, between about 8,500 pg/ml and about 9,000 pg/ml, between about 9,000 pg/ml and about 9,500 pg/ml, or between about 9,500 pg/ml and about 10,000 pg/ml.

In a particular embodiment of the foregoing method, the first amniotic fluid protein fraction comprises an epidermal growth factor, wherein the standardized concentration of the epidermal growth factor is greater than about 20 picograms per milliliter. In a particular embodiment of the foregoing method, the first amniotic fluid protein fraction comprises angiogenin, wherein the standardized concentration of the epidermal growth factor is greater than about 300 picograms per milliliter. In a particular embodiment of the foregoing method, the first amniotic fluid protein fraction comprises monocyte chemoattractant protein-1, wherein the standardized concentration of the epidermal growth factor is greater than about 10 picograms per milliliter. In a particular embodiment of the foregoing method, the first amniotic fluid protein fraction comprises matrix metalloproteinase-1 . In a particular embodiment of the foregoing method, the first amniotic fluid protein fraction comprises matrix metalloproteinase-10, wherein the standardized concentration of the epidermal growth factor is greater than about 300 picograms per milliliter. In a particular embodiment of the foregoing method, the first amniotic fluid protein fraction comprises tissue inhibitor of matrixmetalloproteinase-1, wherein the standardized concentration of the epidermal growth factor is greater than about 4,000 picograms per milliliter. In a particular embodiment of the foregoing method, the first amniotic fluid protein fraction comprises tissue inhibitor of matrixmetalloproteinase-2, wherein the standardized concentration of the epidermal growth factor is greater than about 1,500 picograms per milliliter. In a particular embodiment of the foregoing method, the first amniotic fluid protein fraction comprises hepatocyte growth factor, wherein the standardized concentration of the epidermal growth factor is greater than about 200 picograms per milliliter. In a particular embodiment of the foregoing method, the first amniotic fluid protein fraction comprises insulin-like growth factor binding protein-2, wherein the standardized concentration of the epidermal growth factor is greater than about 6,000 picograms per milliliter.

The invention also includes methods of forming an amniotic fluid derivative that includes the steps of separating an amniotic fluid supernatant from a donor amniotic fluid, separating a protein fraction from a secondary protein source, combining the amniotic fluid supernatant and the protein fraction from the secondary protein source, and diluting the protein fraction from the secondary protein source to a designated concentration with a fluid. In some embodiments, the amniotic fluid supernatant is separated from the donor amniotic fluid by centrifugation. In some embodiments, the protein fraction from the secondary source is separated from the secondary protein source by centrifugation. In some embodiments, the amniotic fluid supernatant is separated from the donor amniotic fluid by any of centrifugation, precipitation, filtration, or a combination thereof. In some embodiments, the protein fraction from the secondary source is separated from the secondary protein source by any of centrifugation, precipitation, filtration, or a combination thereof. In some embodiments, the method further comprises the step of adding a culture media sourced from a stem cell culture, wherein the culture media comprises the secondary protein source.

In some embodiments of the foregoing method, the secondary protein source is any of a cellular lysate, a cellular lysate comprising an amniotic fluid cellular component, or a platelet-rich plasma. In some embodiments of the foregoing method, the protein fraction separated from the secondary protein source comprises a recombinant protein.

The foregoing and other features and advantages of the present invention will be apparent from the following more detailed description of the particular embodiments of the invention, and as illustrated in the accompanying drawings.

### BRIEF DESCRIPTION OF THE DRAWINGS

FIG. 1 is a schematic representation of an embodiment of an amniotic fluid preparation with a standardized biologic activity;

FIG. 2 is a schematic representation of an embodiment of a donor amniotic fluid comprising a supernatant, an exosome component, and a cellular component; and

FIG. 3 is a schematic representation of an embodiment of the inter-relationship between a donor amniotic fluid, a secondary protein source, a first protein fraction, and a second protein fraction.

### DETAILED DESCRIPTION OF THE INVENTION

A detailed description of the described embodiments of the disclosed composition are presented herein by way of examples and not are not limited with reference to the drawings. Although certain embodiments are shown and described in detail, it should be understood that various changes and modifications may be made without departing from the scope of the claims. The scope of the present disclosure will in no way be limited to the number of components, the materials thereof, the shapes thereof, the relative arrangement thereof, etc., and are disclosed simply as examples of embodiments of the present disclosure.

As a preface to the detailed description, it should be noted that, as used in this specification and the appended claims, the singular forms “a”, “an” and “the” include plural referents, unless the context clearly dictates otherwise.

It is to be understood that some of the terms used herein to disclose the elements and various embodiments of the present invention may have broad meaning according to at least the definitions provided herein below. “Amniotic fluid” (abbreviated herein as “AF”) means fluid originating in the amniotic sac of a pregnant female and comprising suspended cellular and non-cellular elements, including all defined and undefined components, molecules, and compounds. “Amniotic-fluid derived” means a substance, compound, cell, cellular organelle, or any other material or structure without limitation sourced from amniotic fluid, regardless of whether other sources of the material are available.

“Preparation” means a substance specially made up from component substances. In an example, an amniotic fluid preparation is a substance specially made up from at least one or more of a group of components comprising a protein fraction and a fluid. This example is not meant to be limiting. “Composition,” as used herein, is synonymous with “preparation.”

“Derivative” means a mixture, solution, composition, material component, and the like, originating from a specific named source. For example, an “amniotic fluid derivative” means a mixture, solution, composition, material component, etc. originating from amniotic fluid.

“Protein fraction” means at least one protein comprising AF. Protein fraction is a portion of an AF supernatant containing a protein. A protein fraction may comprise one protein, a plurality of proteins, or the entire AF proteome. A protein fraction may comprise an additional non-AF protein from a secondary source separate from a donor AF, including an AF protein from a second donor AF, a non-AF protein, or an AF protein or other protein produced outside of AF by other means such as by a genetically engineered bacterium, mammalian cell, or yeast baculovirus, by extracellular in vitro protein synthesis, and the like.

“Biologic activity” means an effect of a substance on a living cell, tissue, or organism. Biologic activity may be quantified directly, such as with an enzyme assay. Biologic activity may otherwise be quantified indirectly, such as with a measurement of the concentration of the biologically active substance, such as by weight or molar amount per unit volume within an AF-derived preparation for example. The aforementioned examples in this paragraph are not meant to be limiting. The biologic activity of a substance within embodiments of an AF-derived preparation may be any quantifiable measurement, whether direct or indirect, of a substance as meaning the biologic activity of the substance.

“Cell” means an intact cell of any origin, and includes a plurality of cells, as used herein. Although extraction of a cell or cells from amniotic fluid is discussed herein, if not otherwise specified a cell may or may not be obtained from amniotic fluid. “Cell type” means a cell that shares one or more common phenotypic characteristics with other cells of the same type. Some examples of shared phenotypic characteristics include expression of cell surface receptor molecules, expression of non-receptor membrane proteins, expression of similar morphology, potential to differentiate into the same more specialized cell or tissue, expression of similar functional characteristics, and the like. “Cellular component” means the entirety of intact cells present in a preparation, a donor amniotic fluid, or other natural or artificial composition. A cellular component may comprise a single cell type, a predominant cell type, or a plurality of cell types, unless otherwise specified herein. In the case of an amniotic fluid preparation, and other preparations, derivatives, and compositions, the cellular component may be suspended within a fluid component. The cellular component includes any cell type, whether defined and known or undefined and unknown, which may be present in AF.

“Cell concentration” means the number of cells present per unit volume of a fluid, such as the number of cells in a milliliter of fluid, for example. “Viable cell concentration” means a cell concentration wherein the counted cells are viable cells wherein viability is determined by a standard dye exclusion assay. A non-limiting example of a dye exclusion assay is a Trypan blue assay; other dye exclusion assays and other methods of determining cell viability may be available. “Designated concentration” means a specific concentration of viable cells in an amniotic fluid-derived preparation that is determined prior to forming an amniotic fluid preparation with a standardized cellular component from an amniotic fluid-derived preparation. The designated cell concentration will vary, depending upon the intended end-use of the amniotic fluid preparation with standardized cellular component, in some embodiments.

“Stem cells” means undifferentiated cells which may give rise to additional generations of stem cells or which may differentiate into progenitor cells. When used in this application, “stem cell” means a stem cell originating in fetal membranes, other fetal-derived tissues, or non-fetal tissues. “Stem cell,” as used herein, includes but is not restricted to stem cells originating in a donor amniotic fluid. “Epithelial stem cell” means a stem cell originating from the embryonic epithelium, including the ectoderm and the endoderm embryonic layers. “Mesenchymal stem cell” means a stem cell capable of lineage differentiation into mesenchymal lineages; for example, osteogenic, chrondrogenic, and adipogenic lineages, and originating from the embryonic mesenchyme, including stromal and vascular tissue of the umbilical cord. “Neural stem cell” means a stem cell capable of lineage differentiation into a central nervous system primary cell type, including a neuron, an astrocyte, and an oligodendrocyte. Wherein “stem cell” is used as referring to a stem cell not originating in the cellular component of AF, the specification will explicitly note a non-AF origin of the stem cell. “Progenitor cell” means a cell which is committed to differentiating 1) along a specific germ cell line, i.e., ectoderm, mesoderm, or endoderm; or 2) a cell committed to differentiating into a specific cell or tissue, i.e., chondrocyte or integrated cortical columnar unit. “Adult stem cell” means a stem cell which may be found among differentiated cells in a tissue or organ of an animal, including a human. “Adult stem cells” may, but are not necessarily, acquired from the tissue of an adult; it is understood that adult stem cells may be found in the tissues of children and infants, as well as adults. “Somatic stem cell,” as used herein, is synonymous with “adult stem cell.”

“Chondroblast” means a progenitor cell of mesenchymal origin which may form a chondrocyte when growing within a collagenous matrix. “Osteoblast” means a terminally-differentiated cell of mesenchymal origin which may form an osteocyte when growing within an osseous matrix. “Neural stem cell” means a self-renewing, pluripotent cell characterized by the ability to terminally differentiate into a neural cell phenotype, such as a neuron, an astrocyte, or an oligodendrocyte under specific micro-environmental conditions found in neural tissues.

“Relative centrifugal force” means the radial force generated by a spinning centrifuge rotor expressed relative to the earth's gravitational force. For example, a relative centrifugal force of 100 g means a radial force one hundred (100) times the force of gravity. “Supernatant” means the liquid layer layered over insoluble material after centrifugation which may be removed, using methods such as pipetting or decanting. The meaning of “supernatant” additionally includes any fluid layered over a solid residue following crystallization, precipitation, or other process causing the solid residue to become distinct from the covering fluid. Supernatant includes water or other liquid and all constituent materials, including compounds in solution or suspension and intact cells, cellular elements, organelles, membrane fragments, and the like remaining in suspension following centrifugation, precipitation, and the like.

“Buffer solution” means an aqueous solution comprising a weak acid and its conjugate base used to stabilize the pH by resisting changes in pH when acid or base is added. A buffer solution is used to stabilize the pH of the solution within a narrow range around a specific value. “Buffer solution” is used generically herein to mean any of many buffer solutions appropriate for a given application and not one specific buffer solution. Unless the use of one specific buffer solution, for example a phosphate buffer solution (“PBS”) such as those commonly used in biologic applications, is explicitly noted herein, the choice of a particular buffer solution for the given use is considered known to one of skill in the art.

“Donor” means a pregnant female, including a peripartum female delivering an infant, from whom amniotic fluid is obtained. “Fetal placental membranes” is used synonymously with “fetal membranes” and means any or all of the amnion, chorion, and Wharton's jelly. “Concentrated” means a relative concentration of a cell, a protein, a non-cellular non-protein substance or other material per unit volume that is greater than the original concentration of that substance in the donor AF. “Substantially depleted” means a concentration of a cell, a protein, a non-cellular non-protein substance, or other material per unit volume of a preparation or fluid wherein the concentration is less than, for example, about 10 percent, less than about 20 percent, less than about 30 percent, less than about 40 percent, or less than about 50 percent of the concentration of that material in the donor AF from which the material derived. Where “substantially depleted” means a specific concentration, this is indicated herein.

“Cellular lysate” means the intracellular products released by the disruption of a cell membrane by any means, such as mechanical, chemical, or other means. “Recombinant” means a protein synthesized from a segment of recombinant DNA, such as a segment of DNA from one organism or species combined with a second segment of DNA from the same or a second organism or species, without limitation. By way of illustration, a segment of human DNA coding for angiogenin is coupled to a strand of bacterial DNA and inserted into a bacterium as a plasmid, wherein the bacterium produces angiogenin through transcription of the recombinant DNA. “Platelet-rich plasma” means blood plasma enriched with platelets. Culture media means a fluid or semi-solid material taken from a cell culture comprising extracellular proteins produced by the cultured cells. “Secondary protein source” or “secondary source” means a source of a protein other than a first donor amniotic fluid. Some non-limiting examples of secondary sources are cellular lysates, genetically recombinant bacteria, cell culture media sourced from a cell culture, platelet-rich plasma, and a second donor amniotic fluid.

“Epidermal growth factor” means any of the protein products encoded by the genomic sequence described in the entry for National Center for Biotechnology Information Gene ID: 1950, including protein precursors, processed proteins, and translated protein products of mRNA transcripts encoded by epidermal growth factor genomic sequences described in that entry.

“Angiogenin” means any of the protein products encoded by the genomic sequence described in the entry for National Center for Biotechnology Information Gene ID: 283, including protein precursors, processed proteins, and translated protein products of mRNA transcripts encoded by angiogenin genomic sequences described in that entry.

“Monocyte chemoattractant protein-1” means any of the protein products encoded by the genomic sequence described in the entry for National Center for Biotechnology Information Gene ID: 6341, including protein precursors, processed proteins, and translated protein products of mRNA transcripts encoded by monocyte chemoattractant protein-1 genomic sequences described in that entry.

“Matrix metalloproteinase-1” means any of the protein products encoded by the genomic sequence described in the entry for National Center for Biotechnology Information Gene ID: 4312, including protein precursors, processed proteins, and translated protein products of mRNA transcripts encoded by matrix metalloproteinase-lgenomic sequences described in that entry.

“Matrix metalloproteinase-10” means any of the protein products encoded by the genomic sequence described in the entry for National Center for Biotechnology Information Gene ID: 4319, including protein precursors, processed proteins, and translated protein products of mRNA transcripts encoded by matrix metalloproteinase-10 genomic sequences described in that entry.

“Tissue inhibitor of matrix metalloproteinase-1” means any of the protein products encoded by the genomic sequence described in the entry for National Center for Biotechnology Information Gene ID: 7076, including protein precursors, processed proteins, and translated protein products of mRNA transcripts encoded by tissue inhibitor of matrix metalloproteinase-1 genomic sequences described in that entry.

“Tissue inhibitor of matrix metalloproteinase-2” means any of the protein products encoded by the genomic sequence described in the entry for National Center for Biotechnology Information Gene ID: 7077, including protein precursors, processed proteins, and translated protein products of mRNA transcripts encoded by tissue inhibitor of matrix metalloproteinase-2 genomic sequences described in that entry.

“Hepatocyte growth factor” means any of the protein products encoded by the genomic sequence described in the entry for National Center for Biotechnology Information Gene ID: 3082, including protein precursors, processed proteins, and translated protein products of mRNA transcripts encoded by hepatocyte growth factor genomic sequences described in that entry.

“Insulin-like growth factor binding protein-2” means any of the protein products encoded by the genomic sequence described in the entry for National Center for Biotechnology Information Gene ID: 3485, including protein precursors, processed proteins, and translated protein products of mRNA transcripts encoded by insulin-like growth factor binding protein-2 genomic sequences described in that entry.

The disclosed invention relates to AF-derived preparations. Specifically, embodiments of the invention comprise preparations derived from AF comprising a standardized biologic activity. The disclosed embodiments of AF-derived preparations with a standardized biologic activity may be used in, for example, but not limited to, tissue regenerative therapy, other medical therapies, and research into the treatment of multiple surgical and non-surgical degenerative conditions.

AF and its constituent components occupy a unique position in the field of regenerative medicine. AF, which derives from both maternal plasma and the developing embryo and fetus, comprises water, electrolytes, proteins and other classes of biologically active molecules, and cells. The cellular component includes epithelial and mesenchymal stem cells of both fetal and maternal origin.

AF may be separated into a cellular component and a supernatant. This separation is commonly accomplished by centrifugation, although other suitable means are available, such as ultrafiltration, precipitation, and the like.

The supernatant contains a large variety and concentration of proteins and other large and small molecules. Each type of biomolecule or family of biomolecules comprises a biological activity. Many of these biomolecules are proteins. In addition to albumin and immunoglobulin, multiple families of regulatory proteins are present which likely affect fetal growth, development, and interaction with the maternal physiologic environment. Growth factors secreted by the mother and fetus are the principal non-cellular active biological compounds native to amniotic fluid. Systematic evaluation of the human amniotic fluid proteome has identified numerous proteins within gene ontology (“GO”) categories relevant to tissue healing, regenerative bioactivity, and biologic augmentation. GO categories are functional identifiers of gene and protein networks that indicate the functional significance of proteins and genes naturally present in amniotic fluid. Key GO categories that have so far been identified include: 1) cellular movement; 2) development and function; 3) cellular growth and proliferation; 4) cell-to-cell signaling and interaction; 5) tissue differentiation; and 6) organism development. These GO classifiers identify the presence of specific categories of growth factors and growth factor networks directly associated with regenerative bioactivity. Growth factors and other compounds present in the AF proteome have hormonal and paracrine activity which exert regenerative effects on both non-AF cells and tissues and AF-derived cells.

The cellular component includes different families of stem cells, of both embryonic and extra-embryonic (maternal) origin. AF stem cells include mesenchymal stem cells. These stem cells are often capable of engraftment and differentiation within host tissue of another individual. AF stem cells are also capable of paracrine secretion of regenerative growth factors and other bioactive substances. Additionally, AF stem cells neither express human leukocyte Class I antigens (“HLA-I”), nor can they differentiate into hematopoietic cells. Consequently, transplanted AF stem cells generally do not provoke an immune response in the recipient and cannot differentiate into host-sensitized T-lymphocytes capable of mounting a graft-versus-host reaction. The lack of immunogenicity makes donor AF-derived stem cells a unique and versatile allograft.

AF also comprises progenitor cells for both hematologic and non-hematologic cell types. Progenitor cells are in a higher state of differentiation than stem cells, although they may still retain the capacity to differentiate into more than one cell type. Also in contrast to stem cells, progenitor cells have a limited capacity for self-renewal spanning only a limited time period, versus possible unlimited self-renewal of stem cells over a time period which may encompass the lifespan of the organism.

AF for amniotic fluid-derived preparations is potentially available in substantial quantities from a pool of donors. There are almost 4 million births per year in the United States, constituting a pool of potential AF donors. From this pool, AF is made available from a suitably screened subpopulation. Potential donors undergo a pre-donation screening process to minimize the risk of transmission of maternal or fetal infectious agents by way of donated AF to an eventual recipient of an amniotic fluid preparation. This screening procedure includes subjective and objective components. The subjective component may include screening by administration of a donor questionnaire to identify high-risk social behaviors for infectious disease. The potential for compensation motivates some paid donors to hide a past social history of high-risk behavior for transmission of sexually transmitted infections, including hepatitis B virus (“HBV”), hepatitis C virus (“HCV”), and human immunodeficiency virus (“HIV”). Accordingly, to avoid unethical behavior that may arise from paid donors, volunteer donors may be used instead. The objective component comprises (pre-delivery) laboratory screening including a metabolic panel including liver function studies and assessment of serology for evidence of past or present HBV, HCV, or HIV infection, in some embodiments.

AF from acceptable donors may be excluded by perinatal indicators, events, and observations. Clinical or laboratory evidence of active maternal or fetal infection around the time of delivery, the most severe example exemplified by chorioamnionitis, precludes the use of AF. Meconium staining of the AF and/or the fetal membranes, although not necessarily indicative of infection, also eliminates the individual from the donor pool. Finally, and most commonly, contamination of the placental membranes with a large quantity of maternal blood, feces, or other perinatal sources of gross bacterial or maternal tissue contamination precludes use of the AF.

Unlike fetal placental membranes, it is generally not practical to obtain AF from a donor during a vaginal delivery. In the majority of vaginal deliveries, the placental membranes spontaneously rupture and the AF is lost. Controlled, therapeutic rupture of membranes, however, is an exception and is discussed herein below. The use of AF from donors undergoing a Cesarean-section delivery essentially eliminates gross bacterial contamination of the donor AF. Of the approximately 4 million births annually in the U.S. mentioned earlier, approximately 33%—1.32 million overall—are by Cesarean delivery. This exclusion of non-Cesarean deliveries; i.e., vaginal deliveries, reduces the potential donor pool for AF by nearly seventy percent. AF, therefore, is potentially available to develop AF-derived preparations from a total between 0.95 and 1.32 million potential donor births annually in the U.S.

As noted herein above, AF may be collected from suitable volunteer donors and processed for storage prior to deriving preparations for use in a variety of clinical applications, both relating to surgical procedures and non-surgical treatments.

Some examples of non-surgical clinical applications for the use of AF-derived preparations include use in dressings and wound treatments as an adjunct to healing, particularly in the treatment of chronically ischemic or infected wounds; as a component in the creation of artificial skin, and to augment healing of tendon and ligamentous injuries. Surgical uses of AF-derived preparations include introduction as an adjunct to healing of surgically repaired bone, tendon, other soft tissue, and open wounds; a means to militate the formation of scar tissue and adhesions, and other beneficial applications in surgery and non-surgical minimally invasive medical therapies. AF-derived preparations may be added to augment biologic dressings, which are commercially available from a variety of sources, with stem cells and growth factors to treat burns, skin pressure ulcers, other chronic open wounds, corneal ulcers, and as a dressing following corneal transplant and other ocular procedures. AF-derived preparations may be used as a component of the extracellular matrix in bioengineered connective tissue scaffolding for tissue and organogenesis using extraembryonic stem cells and other progenitor cells. AF-derived preparations may possess the anti-inflammatory properties of AF and may be used to prevent the development of postoperative adhesions between the tendon, tendon sheath, and associated tissue following tenolysis, synoviolysis, surgical repair of a damaged tendon, and surgical debridement of necrotic or damaged tendon tissue. AF-derived preparations may also be useful to prevent nerve cell death and promote axonal regeneration following early repair of peripheral nerve transections.

An injectable AF-derived preparation allows for use of the composition in both surgical and minimally invasive settings. The injectable AF-derived preparation may be injected into a defined closed space near the end of the surgical procedure, but prior to closing superficial layers of muscle, fascia, and skin at a time when precise placement of the preparation under the surgeon's direct visualization is possible. For example, an injectable AF-derived preparation, depending on the viscosity of the final product, is delivered by injection though a hypodermic needle as small as 30-gauge (“G”) into a closed tendon sheath following tenolysis or tendon repair, into a closed joint capsule following repair of intra-articular cartilage, ligaments, or total joint replacement, into the peritoneal cavity following closure of the abdominal wall, into the pleural space following closure of the chest wall, and into the subdural space following closure of the spinal or intracranial dura mater. An injectable AF-derived preparation of higher viscosity is injected through a 23 G, 22 G, 21 G, 20 G, 18 G, 16 G, or larger-bore hypodermic needle in these and other surgical and minimally invasive applications. An injectable AF-derived preparation of lower viscosity is injected through a 25 G or 30 G needle for use in fine neural repair, aesthetic surgery, and other applications. Following wound closure, an injectable AF-derived preparation may also be re-injected into the defined closed space during the perioperative and postoperative period if deemed useful by the surgeon or other healthcare provider.

An injectable AF-derived preparation may also be injected into a tissue bed in a minimally invasive non-surgical setting. For example, a syringe containing a quantity of the AF-derived preparation is fitted with a hypodermic needle of suitable size for the intended application. The needle is directed to the target tissue bed using visualization and palpation of external landmarks by the provider. Placement of the needle within the target tissue space or tissue may be facilitated with fluoroscopy or other non-invasive imaging modalities. Some examples of minimally invasive uses of AF-derived preparations include intra-articular injection for treatment of injured ligaments, cartilage, and bone; intra-capsular injection of tendon injuries, synovitis, tenosynovitis, and other inflammatory joint conditions; intra-thecal injection for treatment of spinal cord and brain injuries, aseptic meningitis, and other central neurological infections and inflammatory conditions; and other minimally invasive non-surgical applications.

In all of these and other applications, there is strong evidence that the presence of active biomolecules in AF-derived preparations improves healing across a broad range of tissue types, locations within the body, and clinical conditions. Reporting of clinical results may eventually lead to the use of AF-derived preparations as a standard therapy and possibly even the best practice for the treatment of a variety of conditions. Reporting of results requires laboratory experimentation and human clinical trials to generate data for review and interpretation in light of currently available practices and results therefrom. Meaningful interpretation of the generated data, however, depends on reproducibility. Reproducibility requires standardization of materials and techniques. Standardization of AF-derived preparations may include a measure of biologic activity of a particular biologically active sub-component of the AF derived preparation, such as a protein or other biologically active substance.

Standardization of AF-derived preparations may also include a viable cell count present per unit volume of the amniotic fluid preparation. In AF collected from individual donors, substantial differences in both the absolute number and viability of cells in the final preparation will exist based upon the gestational age at collection, other maternal and fetal factors, and preparation methods used.

Preparation, preservation, and packaging of an AF-derived preparation with standardized biological activity may include combination with a cryopreservative, freezing, and/or storage. In some amniotic fluid preparations with standardized biologic activity, a cryopreservative, such as dimethylsulfoxide (“DMSO”), an aqueous glycerol solution, or buffered, balanced electrolyte tissue preservative solution, is added to dilute the AF-derived preparation with standardized biological activity prior to packaging and storage.

What is lacking in the prior art, therefore, is an amniotic fluid preparation incorporating an effective standardized measure of biologic activity of a biologically active substance from an individual donor within the largest possible pool of volunteer donors, packaged and stored to preserve the biological activity of the preparation.

Embodiments of this invention address these and other fundamental requirements of an amniotic fluid preparation—high concentrations of beneficial biomolecules in a standardized preparation with reproducible biologic effects which are preserved throughout packaging, frozen storage, and thawing; essentially no feto-maternal antigenic material, with minimal waste of available donor AF. The amniotic fluid preparation with a standardized biologic activity comprises a donor AF which has been separated into its cellular elements and a supernatant. The supernatant is washed and concentrated to a pre-determined concentration by weight or specific biologic activity using an acceptable fluid to stabilize and preserve biologic activity throughout packaging, lyophilizing, and storage of the amniotic fluid-derived preparation.

Disclosed is an AF-derived preparation comprising a supernatant separated from a donor AF; and a fluid, wherein the fluid dilutes the supernatant to a designated concentration, such as weight per unit volume or biologic activity per unit volume in the AF-derived preparation. Some embodiments of the invention comprise a first protein fraction sub-component of an AF supernatant. Some embodiments comprise a second protein fraction, extracted from an AF fluid supernatant or derived from a secondary source. Some embodiments of the invention comprise additional compounds and formation steps to standardize the biologic effects of the AF-derived preparation and to stabilize and preserve protein structure following freezing, storage, and thawing of the preparation. The AF-derived preparation with standardized biologic activity is for use by medical providers as an injectable fluid or non-injectable gel preparation, either by intraoperative application or injection, non-operative percutaneous injection, or direct application to injured, ischemic, infected, or otherwise damaged tissue. The amniotic fluid preparation with standardized biologic activity is also for use by laboratory researchers as a reproducible source of standardized material for basic science research of the effects of AF-derived preparations on healthy, diseased, and damaged tissue in the field of regenerative medicine, orthopedics, neurology, neurosurgery, gynecologic surgery, and in other clinical, basic medical science, and related scientific disciplines. The use of an AF-derived preparation with standardized biologic activity comprising biocompatible fluids, such as an isotonically balanced buffered electrolyte solution and/or a cryopreservative, maximizes delivery of a wide range of regenerative and similarly beneficial biologic substances within a non-antigenic liquid or gel preparation to the targeted treatment tissue.

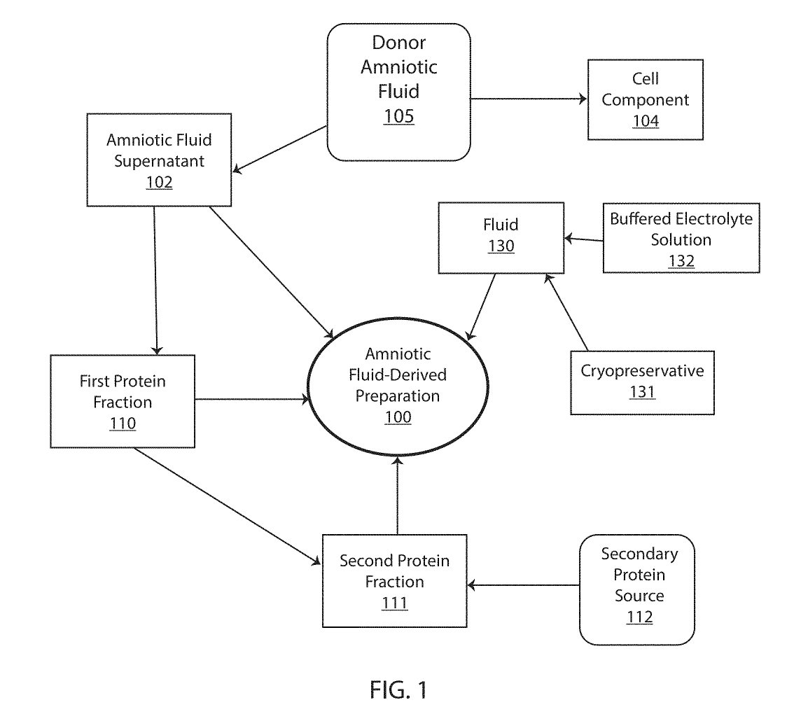

FIG. 1 is a schematic diagram a schematic representation of an AF-derived preparation 100 with a standardized biologic activity. As shown in FIG. 1, AF-derived preparation 100 comprises an AF supernatant 102 and a fluid 130. In some embodiments, AF-derived preparation 100 comprises a first protein fraction 110 extracted from AF supernatant 102. In some embodiments, AF-derived preparation 100 comprises a second protein fraction 111.

AF supernatant 102, in some embodiments, is obtained following collection and separation of a donor AF 105. As shown in FIG. 1, donor AF 105 is separated into a cell component 104 and AF supernatant 102. Separation of donor AF 105 is performed by centrifugation, although other separation methods may also be employed, in some embodiments. Such methods include filtration, ultrafiltration, hydrocyclone separation, ultrasonic separation, gravitational sedimentation, and the like. Centrifugation, an example separation technique utilized to obtain cell 104 and AF supernatant 102, is discussed in further detail herein below.

As shown in FIG. 1, fluid 130 comprises a cryopreservative 131 and a buffered electrolyte solution 132, in some embodiments. In some embodiments, fluid 130 consist of cryopreservative 131. In some embodiments, fluid 130 consists of buffered electrolyte solution 132. In some embodiments, fluid 130 comprises cryopreservative 131 and buffered electrolyte solution 132. In some embodiments, cryopreservative 131 and buffered electrolyte solution 132 are combined to form fluid 130. Cryopreservative 131 and buffered electrolyte solution 132 are also discussed in greater detail herein below.

In some embodiments, donor AF 105 is collected from a volunteer human donor. Accepting AF from volunteer donors while excluding any non-volunteer and paid donors from the donor pool is consistent with internationally well-established tissue donation protocols and reduces the risk of donor-transmitted infection to a recipient of AF-derived preparation with standardized biologic activity 100. Screening of potential volunteer donors, therefore, includes obtaining a comprehensive past medical and social history, complete blood count, liver and metabolic profile, and serologic testing for HBV, HCV, HIV, and other infectious agents, in some embodiments.

In some embodiments, donor AF 105 comprises AF collected from a non-human donor animal. A lack of expression of HLA-1 and HLA-D related (“HLA-DR”) epitopes makes cross-species use of AF preparations possible. In some embodiments, AF preparation with standardized biologic activity 100 comprises donor AF 105 from a non-human donor which is completely de-cellularized during post-collection processing to obtain AF supernatant 102. For example, in some embodiments, donor AF 105 from a non-human donor animal is placed in a centrifuge at 400 g for ten (10) minutes and the resulting supernatant is free of cells and cellular debris. In a second non-limiting example, donor AF 105 from a non-human donor animal is filtered through a filter with a 0.22 micrometer pore size, wherein substantially all cells and cellular debris are removed from donor AF 105.

In some embodiments, donor AF 105 is collected during delivery by Cesarean section. The use of a Cesarean-obtained donor AF 105 to prepare AF-derived preparation with standardized biologic activity 100 is preferable in some embodiments because donor AF 105 collected by Cesarean section is obtained and packaged under strict sterile technique in the operating room, with essentially no microbial contamination. In some embodiments, donor AF 105 is collected into a sterile suction canister liner, following surgical exposure of the intact fetal membranes through a trans-abdominal incision and uterine myotomy, by the surgeon-obstetrician nicking the amniotic membrane and inserting a suction catheter tip into the semi-transparent placental sac under direct vision so as to prevent injury to the infant. Following collection of donor AF 105, which takes approximately five to ten seconds, the baby is delivered by the surgeon-obstetrician. Operating room personnel familiar with sterile technique and tissue handling perform all steps necessary to prepare donor AF 105 for packaging.

The sterile container containing donor AF 105 collected under sterile conditions in the operating room is securely closed and placed in a donor tissue specimen bag. This first specimen bag is then placed within a second bag, which is sealed, labeled, and taken from the operating room for packaging on an ice bath in an insulated container. A patient data sheet containing information regarding the maternal donor is placed in the container, and a separate copy of this information is recorded and logged prior to closing the package. The packaged specimen container is then immediately transported to a processing facility by staff who rotate on call, such that there is minimal delay following delivery before the donor tissue arrives at the separate facility for processing.

Despite the preference for a Cesarean-collected donor AF 105, trans-vaginally collected donor AF 105 is utilized, in some embodiments, to increase the pool of potential donors. In some embodiments, trans-vaginal collection of donor AF 105 is performed in a clinical setting wherein trans-vaginal rupture of fetal membranes is indicated to initiate or promote the progression of labor. Similar sterile collection and handling practices as discussed herein above are utilized, although donor AF 105 is collected with a sterile suction cannula placed through the dilated cervix against the intact fetal membranes prior to rupturing the fetal membranes with an amnion hook or similar instrument. Great care must be afforded the trans-vaginally-collected donor AF 105 to prevent microbial contamination. Trans-vaginally-collected AF is not an acceptable donor AF 105 if there is fecal, blood, or other grossly visible contamination noted in the AF or in proximity to the vagina at the time of collection. Neither a trans-vaginally-collected donor AF 105 nor a Cesarean-collected donor AF 105 is acceptable to form AF-derived preparation with standardized biologic activity 100 if meconium is present in the AF or if there is any visible meconium discoloration or staining of the AF.

FIG. 1 additionally shows a first protein fraction 110 and a second protein fraction 111. Second protein fraction 111 is extracted from a secondary protein source 112, in some embodiments. In some embodiments, second protein fraction is extracted from donor AF 105 (not represented in FIG. 1). In some embodiments, second protein fraction is extracted from AF supernatant 102 (not represented in FIG. 1). First protein fraction 110, in some embodiments, is a constituent protein or proteins of AF supernatant 102. In some embodiments, first protein fraction 110 is the entire AF proteome 103. In some embodiments, first protein fraction 110 comprises substantially a single protein. In some embodiments, angiogenin comprises the majority percentage by weight of all proteins in first protein fraction 110. In some embodiments, epidermal growth factor comprises the majority percentage by weight of all proteins in first protein fraction 110. In some embodiments, monocyte chemoattractant protein-1 comprises the majority percentage by weight of all proteins in first protein fraction 110. In some embodiments, matrix metalloproteinase-1 comprises the majority percentage by weight of all proteins in first protein fraction 110. In some embodiments, matrix metalloproteinase-10 comprises the majority percentage by weight of all proteins in first protein fraction 110. In some embodiments, a tissue inhibitor of metalloproteinase-lcomprises the majority percentage by weight of all proteins in first protein fraction 110. In some embodiments, tissue inhibitor of metalloproteinase-2 comprises the majority percentage by weight of all proteins in first protein fraction 110. In some embodiments, hepatocyte growth factor comprises the majority percentage by weight of all proteins in first protein fraction 110. In some embodiments, insulin-like growth factor binding protein-2 comprises the majority percentage by weight of all proteins in first protein fraction 110. In some embodiments, a biologically active protein molecule extracted from AF supernatant 102 comprises first protein fraction 110.

Second protein fraction 111, in some embodiments, is a constituent of AF supernatant 102 not substantially comprising first protein fraction 110. In some embodiments, second protein fraction 111 is extracted from AF supernatant 102. In some embodiments, second protein fraction 111 is not extracted from AF supernatant 102; rather, second protein fraction 111 is extracted from secondary protein source 112. In some embodiments, second protein fraction 111 is not a constituent of AF supernatant 102.

Secondary protein source 112, in some embodiments, is a cellular lysate. In some embodiments, secondary protein source 112 is a platelet-rich plasma. In some embodiments, secondary protein source 112 is a recombinant organism, such as a genetically recombinant bacterium. In some embodiments, secondary protein source 112 comprises a culture media taken from a stem cell culture. In some embodiments, secondary protein source 112 comprises is derived from a second donor amniotic fluid.

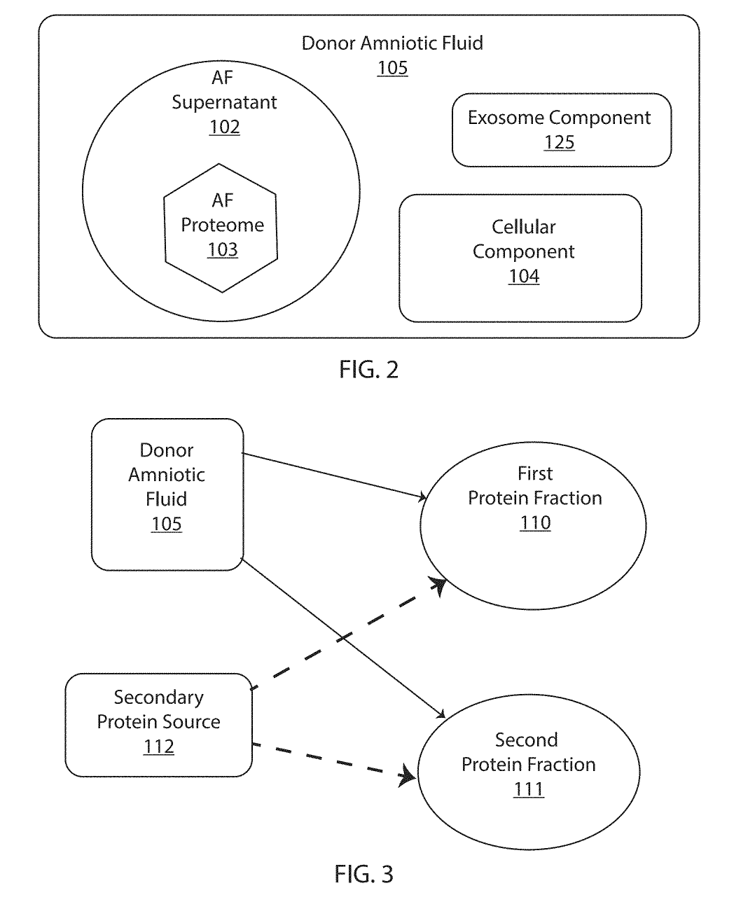

FIG. 2 is a schematic representation of the constituent components of donor AF 105. As shown in FIG. 2, donor AF 105 comprises AF supernatant 102 and a cellular component 104. AF supernatant 102, in turn, comprises an AF proteome 103 and, in some embodiments, a variety of other substances (not shown in FIG. 2), including but not limited to electrolytes, phospholipids, carbohydrates, and urea. “AF proteome 103” means the entire set of products of RNA transcription, protein translation, and post-translational protein modifications manifest as proteins and polypeptides within donor AF 105, the composition of which will vary between individual donor amniotic fluids 105.

Additionally, donor AF 105 comprises an exosome component 125. An “exosome” means a membrane-bounded sub-cellular structure which may comprise proteins, messenger ribonucleic acid, and other biologically active substances. AF, including donor AF 105, along with other body fluids including blood sera and urine, comprise exosomes. Exosome component 125, in some embodiments, is extracted from donor AF 105. In some embodiments, exosome component 125 is added to fluid 130, AF supernatant 102, or first protein fraction 110 to form AF-derived preparation with standardized biologic activity 100. In some embodiments, the exosome component may include one or more of major histocompatibility complex class I or II molecules, cytosolic chaperone proteins, microRNAs (for example, miR-150, miR-142-3p, miR-451, miR-15b, miR-16, miR-196, miR-21, miR-26a, miR-27a, miR-92, miR-93, miR-320, miR-20, let-7a, miR-146a, let-7f, miR-20b, miR-30e-3p, miR-222, miR-6087, miR-126, miR-130a, miR-135b, miR-200a, miR-200b, miR-200c, miR-203, miR-205, miR-141, miR-155, miR-17-3p, miR-106a, miR-146, miR155, miR-191, miR-192, miR-212, miR-214, and miR-210), Rab GTPase, SNAREs, flotillin, subunits of trimeric G proteins, cytoskeletal proteins, annexins, integrins, cholesterol, sphingomyelin, ceramides, hexosylceramides, phosphatidylcholine, phosphatidylethanolamine, phosphatidylserine, elongation factors, Delta 4, syndecan-1, STAT3, PDGF, VEGF, hepatocyte growth factor, sonic hedgehog (SHH), MFGE8, GW182, AGO2, Hsp60, Hsc70, Hsp90, Hsp20, 14-3-3 epsilon, PKM2, nuclear factor κB (NFκB), tetraspanins, CD9, CD63, CD80, CD86, CD19, CD81, CD82, CD53, CD37, CD34, CD41, CD62p, TSG101, matrix metalloproteinases (MMPs), extracellular matrix metalloproteinase inducer, AU rich element binding proteins (e.g., KRSP and TTP), RNA binding proteins (e.g., MPP6 and C1D), Rrp40, hCs14, hRrp4, hRrp40, PM/Scl-75, Dis3, Dis3L1, Rrp44-H1, Rrp44-H2, Rrp44-H3, hRrp41, hRrp42, hMtr3, hRrp43/OIP2, hRrp46, PM/Scl-100, or any combination thereof.

In some embodiments, the non-cellular components of donor AF, including AF proteome 103, are separated from cellular component 104 by centrifugation, as mentioned herein above. Centrifugation involves the use, in some embodiments, of commercially available equipment and established techniques known in the art. For example, in some embodiments, donor AF 105 is centrifuged at a relative centrifugal force (“RCF”) of between 300 g and 500 g for ten (10) minutes. At this speed and duration, the supernatant is essentially cell free, with all cells and cellular debris from donor AF 105 present in the pellet. Other non-limiting examples of centrifugation utilize RCFs from 300 g to 1000 g for a duration from three (3) and ten (10) minutes. In some embodiments, AF 105 is centrifuged at an RCF of less than 300 g for between five (5) and ten (10) minutes. In some embodiments, AF 105 is centrifuged at an RCF greater than 1000 g. In some embodiments, AF 105 is centrifuged for greater than ten (10) minutes. The choice of the force (RCF) and duration of AF centrifugation will depend, in some embodiments, upon factors such as the mechanical fragility characteristics of specific cells to be retained as viable cells 104 in amniotic fluid-derived preparation with standardized biologic activity 100.

Following centrifugation, the water-based AF supernatant 102 comprises AF proteome 103 and a variety of other substances not shown in the figures, including electrolytes, phospholipids, carbohydrates, and urea. Depending upon the intended specific therapeutic use of amniotic fluid-derived preparation with standardized biologic activity 100, it may be desirable for AF supernatant 102 to be depleted of one or more components of a group of components comprising individual electrolytes, phospholipids, carbohydrates, urea, and the like present in AF supernatant 102. For example, amniotic fluid-derived preparation with standardized biologic activity 100 with cellular component 104 comprising mesenchymal stem cells is depleted of phospholipids and urea, in some embodiments. Consequently, a concentration method is used, in some embodiments, to form supernatant 102 comprising a concentrated protein fraction, for example. Concentrating a protein or other fraction of such components found in AF supernatant 102 is accomplished by a variety of methods. Alternatively, in some embodiments, supernatant 102 is depleted of a protein or other substance using one of the methods described herein below. Dialysis against a solution of a defined composition is a relatively simple and efficient method to deplete or otherwise manipulate the concentrations of individual components comprising AF supernatant 102. This is a non-limiting example; other techniques, such as chromatography, may also be used.

Conversely, in some embodiments, supernatant 102 comprises proteome 103 depleted of a constituent protein by precipitation. Otherwise stated, rather than incorporating the entire proteome 103 in supernatant 102, a protein is removed from supernatant proteome 103 by precipitation, leaving supernatant 102 comprising the remaining constituent proteins of proteome 103. In some embodiments, the depleted protein is albumin. In some embodiments, the depleted protein is immunoglobulin.

In some embodiments, an individual protein or group of proteins is separated from proteome 103 using a density-gradient centrifugation technique. In one non-limiting example protocol, 100 microliters (0.1 milliliters) of AF supernatant 102 is layered onto a sucrose gradient solution in a centrifuge tube, the gradient comprising (from bottom of the tube to the liquid surface) 950 microliters of 40% sucrose solution; 950 microliters of 31.25% sucrose solution; 950 microliters of 22.5% sucrose solution; 950 microliters of 13.75% sucrose solution; and 950 microliters of 5% sucrose solution. The sucrose gradient should be refrigerated at 4° C. for twelve (12) to sixteen (16) hours to allow a linear gradient to form prior to layering AF supernatant 102 and centrifuging. The tube is then centrifuged at approximately 237,000 g for four (4) hours. The tube is removed from the centrifuge and placed in an ice bath. In some embodiments, the protein fractions within a microcentrifuge tube are precipitated by adding 300 microliters (an equal volume) of trichloroacetic acid to the microcentrifuge tube containing the protein fraction and the tube is placed on ice for thirty (30) minutes. The tube is then centrifuged at 15,000 g for fifteen minutes at 4° C. The supernatant is separated, such as by pipetting or decanting techniques. The protein pellet is re-suspended in 100 microliters of buffered electrolyte solution, such as PBS, for example. In this example, and some other embodiments, first protein fraction 110 comprises the resulting re-suspended protein pellet suspension.

Precipitation using ammonium chloride or other suitable compound to refine the protein composition of AF supernatant 102 is by way of example only. Other methods known and practiced in the art, such as liquid chromatography, ultrafiltration-centrifugation, ligand-antibody affinity binding with magnetic separation, and the like may be utilized. The choice of method and details of the procedure wherein AF supernatant 102 is refined are determined by the physiochemical and immunologic characteristics of the specific proteins comprising AF supernatant 102 or to be removed from AF supernatant 102.

Additional quantities of desirable proteins, including proteome 103 proteins, from an individual donor are produced, in some embodiments, by extracting constituent intracellular protein(s) from the cellular component 106 of donor AF 105 separated from AF supernatant 102. In some embodiments, intracellular proteins are extracted from AF collected from a separate donor processed to create a cellular lysate. In some embodiments, for example, the cellular “pellet” obtained following centrifugation of a donor AF 105 is “washed” by re-suspending the pellet in a buffer solution followed by re-centrifugation and removal of the supernatant comprising the buffer solution one or more times. The washed pellet comprising cell component 104 is re-suspended in a quantity of buffer solution to form a cellular suspension in buffer of cell component 104. An aliquot of this suspension is removed and the cells in the aliquot are disrupted by using an established technique known in the art, releasing high concentrations intracellular proteins into the suspension. Non-limiting examples of such techniques include serial freezing-and-thawing, use of detergents, sonication, high pressure filtration, or treatment with organic solvents to disrupt the cell membrane releasing membrane receptors and other membrane proteins.

For example, an aliquot of the cellular suspension is further washed through two suspension/centrifugation cycles with phosphate buffered saline (“PBS”) and the washed cells are placed in culture dishes, on ice. To each dish is added 1.0 milliliter of a detergent lysis buffer, such as a 0.01%-0.05% aqueous solution of sodium dodecyl sulfate or nonylphenoxypolyethoxylethanol (“NP-40”). A commercially available lytic reagent, such as Mammalian Protein Extraction Reagent (“M-PER”) available from Thermo Fisher Scientific of Waltham, Massachusetts, for example, may also be used. The cells are then incubated on ice for between ten (10) and thirty (30), minutes, periodically rocking the dishes gently. A dish is then tilted slightly on the ice bed to allow the buffer solution containing the cellular lysate to drain to one side, where it is removed with a pipette. The pipetted lysate is centrifuged at 20,000 g for ten (10) minutes at 4° C. The supernatant is carefully removed to a fresh centrifuge tube, taking care not to disturb the debris pellet. The lysate may be stored on ice, or flash-frozen using a dry ice/ethanol mixture and then stored at minus seventy degrees Celsius (−70° C.). The foregoing procedure is offered only by way of example and is not intended to be liming.

Alternatively, after cellular disruption, proteins are extracted and purified from the resulting cellular lysate by use of another aforementioned technique under protocols known in the art; non-limiting examples including precipitation, immunoprecipitation, centrifugation on a sucrose, Percoll®, or alternative density gradient; protein electrophoresis; chromatography; fluorescent or magnetic bead-based immunoaffinity separation; other aforementioned non-limiting examples, and the like; in some embodiments.

The resulting purified proteins, cellular lysate, or specific protein(s) are then added to supernatant 102.

FIG. 3 is a schematic representation of the inter-relationship between a donor amniotic fluid 105, a secondary protein source 112, a first protein fraction 110, and a second protein fraction 111. As discussed herein above, first protein fraction 110 and second protein fraction 111 each represents a protein fraction substantially comprising a specific named protein, such as angiogenin, matrix metalloproteinase, insulin-like growth factor binding protein-2, and the like. Regardless of the identity of the primary constituent protein of first protein fraction 110 or second protein fraction 111, first protein fraction 110 and second protein fraction 111 may be derived from either donor AF 105 or from a secondary source 112, including but not limited to the various examples already mentioned herein.

In some embodiments, density-gradient centrifugation within a sucrose solution or a colloidal silica suspension, such as Percoll®, for example, is employed to separate the heterogeneous proteins comprising first protein fraction 110 or second protein fraction 111 into a number of protein fractions based upon the buoyant density of the constituent proteins comprising first protein fraction 110 and second protein fraction 111. During centrifugation, proteins will “band” on the gradient in levels corresponding to the relative buoyant density of each protein fraction. The region containing the desired protein to comprise first protein fraction 110 or second protein fraction 111, for example, is removed from the banded supernatant. Conversely, a region not comprising first protein fraction 110 or second protein fraction 111 is removed, in some embodiments.

In some embodiments, magnetized polymer microbeads, such as Dynabeads®, are reversibly coupled to a specific protein by a monoclonal receptor antibody reversibly bound to an epitope on a specific protein. In some embodiments, an AF supernatant 102 comprising angiogenin is separated and removed from AF supernatant 102 obtained from donor AF 105 by utilizing magnetized polymer microbeads coupled to monoclonal antibodies to angiogenin. The magnetized polymer microbeads bound to the monoclonal antibodies are reacted with a suspension of AF supernatant 102, wherein the monoclonal antibodies bind to epitopes of angiogenin. The suspension of AF supernatant 102 comprising the antigen-antibody bound complexes is then passed through a flow channel within a magnetic field, utilizing a MACS protocol known and practiced in the art, wherein the angiogenin is removed from AF supernatant 102 to form first protein fraction 110.

Following separation from AF supernatant 102, or other source such as secondary source 112, first protein fraction 110 is diluted with a suitable buffer solution. A resulting concentration of a protein comprising first protein fraction 110 per unit volume within the buffer solution is determined using techniques known in the art, in some embodiments. The suspension of first protein fraction 110 is further diluted to a biologic activity per unit volume by adding a volume of additional buffer solution necessary to achieve the biologic activity. As mentioned herein above, biologic activity means concentration by weight, molar concentration, enzymatic activity, or similar direct or indirect measurement of the specific protein of interest comprising first protein fraction 110. In some embodiments, second protein fraction 111 is diluted to a desired unit of biologic activity per unit volume using a suitable buffer solution. In some embodiments, second protein fraction 111 is diluted to a desired measure of biologic activity per unit volume using a suitable buffer solution. In some embodiments, first protein fraction 110 is combined with second protein fraction 111, along with fluid 130 to form AF-derived preparation 100.

AF-derived preparation with standardized biologic activity 100 is formed by combining AF supernatant 102 or first protein fraction 110 and fluid 130. Fluid 130, in some embodiments, comprises buffered electrolyte solution 132, cryopreservative 131, another non-cytotoxic fluid, or any combination thereof.

In some embodiments, buffered electrolyte solution 132 comprises “Plasma-Lyte A,” manufactured by Baxter International, Inc., Deerfield, Ill. This is by example and not meant to be limiting. The composition of buffered electrolyte solution 132 to create an optimal pH and buffering range is chosen according to the specific kinetic, ionic, tertiary and quaternary structure, and other physiochemical characteristics of the enzyme, other protein, or other biologically active molecule(s) comprising AF supernatant 102, first protein fraction 110, or second protein fraction 111 against which the standardized biologic activity is measured. Accordingly, many appropriate compositions of buffered electrolyte solution 132 are possible and may be chosen by one with skill in the biochemical arts.

In some embodiments, cryopreservative 131 comprises CryoStor® CS-10, a 10% solution of dimethylsulfoxide (“DMSO”) manufactured by BioLife Solutions, Inc., Bothel, Wash. In some embodiments, cryopreservative 131 comprises a 5% solution of DMSO. These examples are not meant to be limiting, other examples of non-cytotoxic cryoprotectant fluids may be used, at similar or different concentrations.

Following combination of first protein fraction 110 and second protein fraction 111, a final measurement of biologic activity per unit volume of amniotic fluid-derived preparation 100 is calculated, in some embodiments. To reach a desired final cell count of biologic activity per unit volume of amniotic fluid-derived preparation with standardized biologic activity 100, a required amount of fluid 130 to achieve the desired dilution and this amount is subsequently added to amniotic fluid preparation with standardized biologic activity 100.

In some embodiments, a small quantity of amniotic fluid preparation with standardized biologic activity 100 is drawn into a sterile 2 cc syringe and extruded through a 25 gauge needle to ensure amniotic fluid preparation with standardized biologic activity 100 is sufficiently fluid to be percutaneously or intraoperatively injected into a recipient tissue bed. In some embodiments, the final cell concentration and/or the final biologic activity is adjusted by adding additional fluid 130 to an end-user's pre-ordered requirements based upon the intended use of amniotic fluid preparation with standardized biologic activity 100. In some embodiments, a final cell concentration is adjusted by adding additional fluid 130 to an end-user's pre-ordered requirements based upon the intended use of amniotic fluid preparation with standardized biologic activity 100.

It is useful to employ amniotic fluid preparations of different viscosities for clinical use with knowledge of expected results based upon reproducibility. Variations in viscosity may affect the tendency of amniotic fluid preparation with standardized biologic activity 100 to remain and engraft a fraction of the cell component 104 at the site of placement. Differences in viscosity are considered based upon the intended use of amniotic fluid preparation with standardized biologic activity 100. Some embodiments of amniotic fluid preparation with standardized biologic activity 100 are formed in three reproducible, standardized viscosities: high viscosity; medium viscosity; and low viscosity. Consequently, in some embodiments, the viscosity of amniotic fluid preparation with standardized biologic activity 100 is adjusted by mixing an additional measured quantity of fluid 130 with the amniotic fluid preparation having a standardized biologic activity 100 and calculating the final biologic activity per ml accordingly.

In some embodiments wherein high viscosity amniotic fluid-derived preparation with standardized biologic activity 100 is desired, a measured quantity of biologic “thickening agent” is added to increase the viscosity of formed amniotic fluid-derived preparation with standardized biologic activity 100. In some embodiments, an aqueous “hydrogel” is added do amniotic fluid=derived preparation with standardized biologic activity 100, such as alginate, hyaluronic acid, gelatin, and the like.

High-viscosity amniotic fluid-derived preparation with standardized biologic activity 100 has a measured viscosity of greater than 10,000 centipoise (“cP”) and is formed by adding a biologically compatible thickening agent to amniotic fluid-derived preparation with standardized biologic activity 100. High-viscosity amniotic fluid-derived preparation with standardized biologic activity 100, in some embodiments, is a solid gel. In some embodiments, high-viscosity amniotic fluid-derived preparation with standardized biologic activity 100 is a very thick fluid which is a fluid thicker than about the thickness of honey. Some examples of applications where high-viscosity amniotic fluid-derived preparation with standardized biologic activity 100 may be used include the non-invasive or minimally-invasive treatment of entero-cutaneous, entero-vaginal, entero-enteric, broncho-pleural, tracheal-esophageal fistulas; graft-repair of osteochondral defects in the knee, hop, ankle, wrist, hand, and other joints; microfractures and small facial fractures; and seeding of a biocompatible extracellular scaffold for filling of large bone tissue voids following trauma, ischemic or radiation necrosis, congenital abnormalities, and surgical treatment of certain cancers.

Medium-viscosity amniotic fluid-derived preparation with standardized biologic activity 100 has a measured viscosity of between 100 and 10,000 cP and is formed by adding a biologically compatible thickening agent to amniotic fluid-derived preparation with standardized biologic activity 100. In some embodiments, medium-viscosity amniotic fluid preparation with standardized biologic activity 100 is a fluid with a thickness between about the thickness of motor oil and about the thickness of honey. Examples of applications where medium-viscosity amniotic fluid-derived preparation with standardized biologic activity 100 may be used include treatment of wound sinus tracts, grafting of cutaneous and soft-tissue defects resulting from deep thermal or radiation burns; spinal and other bony fusion procedures (when combined with currently available bone putty or as a stand-alone application into a cervical or lumbar intervertebral spacer); facial trauma and facial fracture treatment; bone grafting; alveolar cleft (“cleft palate”) grafting; treatment of dental/tooth tissue defects; chronic inflammatory bursitis; intervertebral facet-based pain; tears of the meniscal cartilage; application to entero-entero and other surgical anastomoses; treatment of non-union and mal-union of fractures, intra-peritoneal application following surgical adhesiolysis; intra-peritenon implantation following Achilles' tendon debridement and anastamotic repair; defects of the calvarium following trauma; emergency decompressive craniotomy; surgical breast reconstruction; and following acetabular and other articular joint surface resurfacing, for example.

Low-viscosity amniotic fluid-derived preparation with standardized biologic activity 100 has a measured viscosity of less than 100 cP and is formed, in some embodiments, by adding a biologically compatible thickening agent to amniotic fluid-derived preparation with standardized biologic activity 100. In some embodiments, low-viscosity amniotic fluid-derived preparation with standardized biologic activity 100 is formed by adding additional fluid 130 to amniotic fluid-derived preparation with standardized biologic activity 100. Low-viscosity amniotic fluid-derived preparation with standardized biologic activity 100 may be easily injected through a hypodermic needle larger than 25 G and is, therefore, useful in clinical applications wherein preparation 100 is delivered to the target tissue site by injection. Examples of applications where low-viscosity amniotic fluid-derived preparation 100 may be used include treatment of chronic wounds, radiation burns, and thermal injury by subcutaneous injection; injection into peri-rotator cuff soft tissues following rotator cuff repair; injection to facilitate non-surgical repair and healing of supraspinatus, infraspinatus, teres minor, and subscapularis tears; other muscle, ligament, tendon, and soft-tissue tears; epicondylitis; and other similarly debilitating chronic fascial inflammatory conditions such as plantar fasciitis or fasciolosis.

In some embodiments, amniotic fluid-derived preparation with standardized biologic activity 100 is packaged with standardized ranges of any one quantity or combination of quantities of first protein fraction 110, second protein fraction 111, and degree of viscosity based upon the mode used for delivery (injection versus intraoperative application, recipient host tissue type, other specific requirements, for example) and intended therapeutic use.

The completed amniotic fluid-derived preparation 100 is sealed in packaging vials and frozen for storage at minus eighty (−80) degrees Celsius, in some embodiments.

### INCORPORATION BY REFERENCE

The entire disclosure of each of the patent documents and scientific articles cited herein is incorporated by reference for all purposes.

### EQUIVALENTS

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, and are intended to fall within the scope of the appended claims. The disclosure can be embodied in other specific forms without departing from the essential characteristics thereof. The scope of the disclosure is indicated by the appended claims rather than by the foregoing description, and all changes that come within the meaning and range of equivalency of the claims are intended to be embraced therein.

1. An amniotic fluid preparation comprising:
a supernatant separated from a donor amniotic fluid; and
a fluid,
wherein the fluid dilutes the supernatant to a standardized biologic activity in the amniotic fluid preparation.

2. The amniotic fluid preparation of claim 1, further comprising a cellular lysate.

3. The amniotic fluid preparation of claim 1, further comprising a recombinant protein.

4. The amniotic fluid preparation of claim 1, further comprising a platelet-rich plasma.

5. The amniotic fluid preparation of claim 1, further comprising a stem cell culture medium.

6. A set of amniotic fluid preparations, wherein each amniotic fluid preparation in the set is an amniotic fluid preparation of claim 1, and each amniotic fluid preparation in the set has the same or about the same standardized biologic activity as every other amniotic fluid preparation in the set.

7. The set of claim 6, wherein the set comprises at least 10 amniotic fluid preparations or at least 50 amniotic fluid preparations.

8. (canceled)

9. An amniotic fluid preparation comprising:
a first protein fraction separated from a donor amniotic fluid; and
a fluid,
wherein the fluid dilutes the first protein fraction to a standardized concentration in the amniotic fluid preparation.

10. The amniotic fluid preparation of claim 9, wherein the first protein fraction comprises an epidermal growth factor.

11. (canceled)

12. The amniotic fluid preparation of claim 9, wherein the first protein fraction comprises angiogenin.

13. (canceled)

14. The amniotic fluid preparation of claim 9, wherein the first protein fraction substantially comprises monocyte chemoattractant protein-1.

15. (canceled)

16. The amniotic fluid preparation of claim 9, wherein the first protein fraction substantially comprises matrix metalloproteinase-1.

17. The amniotic fluid preparation of claim 9, wherein the first protein fraction substantially comprises matrix metalloproteinase-10.

18. (canceled)

19. The amniotic fluid preparation of claim 9, wherein the first protein fraction substantially comprises tissue inhibitor of matrixmetalloproteinase-1.

20. (canceled)

21. The amniotic fluid preparation of claim 9, wherein the first protein fraction substantially comprises tissue inhibitor of matrix metalloproteinase-2.

22. (canceled)

23. The amniotic fluid preparation of claim 9, wherein the first protein fraction substantially comprises hepatocyte growth factor.

24. (canceled)

25. The amniotic fluid preparation of claim 9, wherein the first protein fraction substantially comprises insulin-like growth factor binding protein-2.

26. (canceled)

27. A set of amniotic fluid preparations, wherein each amniotic fluid preparation in the set is an amniotic fluid preparation of claim 9, and the standardized concentration of the first protein fraction of each amniotic fluid preparation in the set is the same or about the same as the standardized concentration of the first protein fraction of every other amniotic fluid preparation in the set.

28. The set of claim 27, wherein the set comprises at least 10 amniotic fluid preparations or at least 50 amniotic fluid preparations.

29. (canceled)

30. An amniotic fluid derivative comprising:
a supernatant separated from a donor amniotic fluid;
a protein fraction separated from a secondary protein source; and
a fluid,
wherein the fluid dilutes the protein fraction to a designated concentration in the amniotic fluid preparation.

31. The amniotic fluid derivative of claim 30, wherein the secondary protein source is a cellular lysate.

32. The amniotic fluid derivative of claim 31, wherein the cellular lysate comprises an amniotic fluid cellular component.

33. The amniotic fluid derivative of claim 30, wherein the secondary protein source is a platelet-rich plasma.

34. The amniotic fluid derivative of claim 30, wherein the protein fraction comprises a recombinant protein.

35. The amniotic fluid derivative of claim 30, further comprising a culture media sourced from a stem cell culture, wherein the culture media comprises the secondary protein source.

36. A set of amniotic fluid derivatives, wherein each amniotic fluid derivative in the set is an amniotic fluid derivative of claim 30, and the designated concentration of the protein fraction from the secondary protein source of each amniotic fluid derivative in the set is the same or about the same as the designated concentration of the protein fraction from the secondary protein source of every other amniotic fluid derivative in the set.

37. The set of claim 36, wherein the set comprises at least 10 amniotic fluid derivatives or at least 50 amniotic fluid derivatives.

38. (canceled)

39. A method of forming an amniotic fluid preparation, the method comprising the steps of:
separating an amniotic fluid supernatant from a donor amniotic fluid; and
diluting the amniotic fluid supernatant to a standardized biologic activity with a fluid.

40-44. (canceled)

45. A method of forming an amniotic fluid preparation, the method comprising the steps of:
separating an amniotic fluid protein fraction from a donor amniotic fluid; and
diluting the amniotic fluid protein fraction to a standardized concentration with a liquid.

46-63. (canceled)

64. A method of forming an amniotic fluid derivative, the method comprising the steps of:
separating an amniotic fluid supernatant from a donor amniotic fluid;
separating a protein fraction from a secondary protein source;
combining the amniotic fluid supernatant and the protein fraction from the secondary protein source; and
diluting the protein fraction from the secondary protein source to a designated concentration with a fluid.

65-71. (canceled)
